(19) United States
(12) Patent Application Publication
(10) Pub. No.: US 2007/0140897 A1
(43) Pub. Date: Jun. 21, 2007

(54) PH STABLE BIGUANIDE COMPOSITION AND METHOD OF TREATMENT AND PREVENTION OF INFECTIONS

(76) Inventors: Hongna Wang, Fairport, NY (US); Erning Xia, Penfield, NY (US); Srini Venkatesh, Pittsford, NY (US)

Correspondence Address: Bausch & Lomb Incorporated, One Bausch & Lomb Place, Rochester, NY, 14604-2701, US

(21) Appl. No.: 11/614,504

(22) Filed: Dec. 21, 2006

Related U.S. Application Data
(60) Provisional application No. 60/752,455, filed on Dec. 21, 2005.
(60) Provisional application No. 60/760,510, filed on Jan. 20, 2006.
(60) Provisional application No. 60/760,880, filed on Jan. 20, 2006.
(60) Provisional application No. 60/782,478, filed on Mar. 15, 2006.
(60) Provisional application No. 60/830,319, filed on Jul. 12, 2006.
(60) Provisional application No. 60/830,326, filed on Jul. 12, 2006.

(51) Int. Cl. A61L 2/18

(52) U.S. Cl. 422/28

(57) ABSTRACT

The present invention includes an ophthalmically acceptable composition for use in the ocular region of a patient, the ophthalmically acceptable composition comprising water, a biguanide containing antimicrobial agent and a pH adjusting agent to adjust the pH to a minimum of 4 and a maximum of 6. The invention further comprises administering the ophthalmically acceptable composition to the eye of a patient in need of treatment.

### CROSS-REFERENCE TO RELATED APPLICATIONS

This application claims priority to U.S. Provisional Patent Application Nos. 60/752,455 filed Dec. 21, 2005; 60/760,510 filed Jan. 20, 2006; 60/760,880 filed Jan. 20, 2006; 60/782,478 filed Mar. 15, 2006; 60/830,319 filed Jul. 12, 2006 and 60/830,326 filed Jul. 12, 2006; the contents of each being incorporated by reference herein.

### BACKGROUND OF THE INVENTION

1. Field of the Invention

This invention relates to the treatment of ocular infections.

2. Discussion of the Related Art

Ocular infections are conditions that require treatment. Depending on the structures which are involved and the infecting organism, ocular infections may range from discomfort (conjunctivitis) to serious pain and vision loss (keratitis). Ocular infections include bacterial, viral, fungal and amoebal species.

A clinically effective antimicrobial agent is one that is potent against a particular microbe yet is not toxic to human tissue.

Such an agent that is toxic against a wide range of microbes, yet has relatively little toxicity against human tissue is considerably more valuable.

Biguanide antimicrobial agents have been used to preserve ophthalmic solutions and demonstrate relatively low toxicity in ocular tissues. Biguanide antimicrobial agents include polyhexamethylene biguanide, chlorhexidine and Alexidine.

To effectively preserve an ophthalmic composition, sufficient preservative is necessary to prevent growth of S. aureus, P. aeruginosa and E. coli bacteria and C. albicans and A. niger fungi over the shelf life of the product. Typically, a clinically effective formulation will contain the lowest amount of a preservative required to accomplish the desired effect. Between 0.5 ppm and 3.0 ppm of a biguanide has been used to preserve most ophthalmic solutions.

Biguanide antimicrobial agents have been used as disinfectant solutions for contact lenses. To be considered a disinfectant, a solution needs sufficient antimicrobial agent to kill S. aureus, P. aeruginosa and S. marcescens bacteria and C. albicans and F. solani fungi over the shelf life of the product. Furthermore, the solution must show efficacy in disinfecting contact lenses using the disinfecting regimen that is recommended on the product. This regimen is arrived at through data which supports the disinfecting properties described above.

Disinfecting solutions containing antimicrobial agents include ReNu® Multiplus sold by Bausch & Lomb, Rochester, N.Y. ReNu® Multiplus is a multipurpose cleaning, conditioning and disinfecting solution for contact lenses that contains 1 ppm of polyhexamethylene biguanide.

Disinfecting solutions such as the one mentioned above are ophthalmically safe solutions. They are safe to administer to the eye of a patient. Contact lenses that have been rinsed with these solutions are placed in the eye. However, these solutions are not approved for use as a medicament in the eye. There is no evidence to suggest that the level of antimicrobial agent in a multipurpose contact lens solution would be effective to treat ocular infection.

Several studies have been conducted on the effectiveness of polyhexamethylene biguanide and/or chlorhexidine for treatment of Acanthamoebal keratitis and Fungal keratitis.

In Schuster, et al., “Opportunistic Amoebae: Challenges In Prophylaxis And Treatment,” Drug Resistance Updates: Reviews And Commentaries In Antimicrobial And Anticancer Chemotherapy, vol. 7(1) pp. 41-51 (February 2004), Acanthamoeba keratitis, a non-opportunistic infection of the cornea, was found to respond to treatment with chlorhexidine gluconate and polyhexamethylene biguanide, in combination with propamidine isothionate (Brolene), hexamidine (Desomodine), or neomycin.

In Rama, et al., “Bilateral Acanthamoeba keratitis with late recurrence of the infection in a corneal graft: a case report,” European Journal of Ophthalmology, vol. 13 (3), pp. 311-4(April 2003), recurrences of Acanthamoeba keratitis in both eyes were successfully treated with a combination of hexamidine and neomycin, and with polyhexamethylene biguanide, respectively.

Panda, et al., “Role of 0.02% polyhexamethylene biguanide and 1% povidone iodine in experimental Aspergillus keratitis,” Cornea, vol. 22 (2), pp. 138-41, (March 2003) showed that polyhexamethylene biguanide (0.02%) is a moderately effective drug for experimental Aspergillus keratitis.

Fischella, et al. “Polyhexamethylene Biguanide (PHMB) in the Treatment of Experimental Fusarium Keratomycosis,” Cornea, vol. 16(4), pp. 447-49 (1997) teaches that a 0.02% solution of PHMB was effective at reducing fungal growth in a rabbit model.

Sharma, et al., “Patient characteristics, diagnosis and treatment of non-contact lens related Acanthamoeba keratitis,” British Journal of Ophthalmology, vol. 84/10, pp. 1103-1108 (2000) illustrates the combination of polyhexamethylene biguanide and chlorhexidine. See also Alexandrakis, et al., “Amebic Keratitis Due to Vahlkampfia Infection Following Corneal Trauma,” Arch. Ophthalmology, vol. 116, pp. 950-51 (July 1998); Hariminder, et al., “Non-Acanthamoeba Amebic Keratitis,” Cornea, vol. 17(6), pp. 675-677 (1998); Bobo, et al., “Les Keratites Amibiennes,” Med. Trop., vol. 55 (4 bis), pp. 439-443 (1995)(English Abstract); Burger et al., “Acantamoeben-keratitis: Een erstige ooginfectie in optomst,” Pharamceutisch Weekblad, vol. 131(3), pp. 72-77 (1996)(English Abstract); Prajna et al., Effect of Topical 2% Polyhexamethylene Biguanide on Nocardial Keratitis Associated with Scleritis,” Indian Journal of Ophthalmology, vol. 46(4), pp. 251-52 (December, 1998); Messick, et al., “In-vitro activity of polyhexamethylene biguanide (PHMB) against fungal isolates associated with infective keratitis,” J. Antimicrob. Chemother., vol. 44, pp. 297-298 (1999).

Alexidine in addition to polyhexamenthylene biguanide and chlorhexidine was shown to have activity against acantamoeba keratitis at a minimum inhibitory concentration of 6.3 ug/ml for cysts and trophazoites. Pyott, et al., “Acanthamoeba keratitis: first recorded case from a Palestinian patient with trachoma,” British Journal of Ophthalmology, vol. 80, p. 849 (1996). In Conner, et al., “Guanidines, Diamidines and Biguanides: Towards a Rational Therapy for Acanthamoeba Keratitis,” J. Pharm. Pharmacol., vol. 47(12) pp. 1007 indicates that biguanides, including chlorhexidine, alexidine and poly hexamethylene biguanide were considered favorable for first line treatment of Acanthamoeba Keratitis. Chlorhexidine was considered the most preferred. See also, Seal, “Acanthamoeba keratitis update—incidence, molecular epidemiology and new drugs for treatment, Eye, vol. 17, pp. 893-905 (2003).

WO 97/00076 discloses a composition that contains poly(hexamethylene biguanide salts. The formulations include buffers such as borate/boric acid, phosphate buffer, and acetate/citrate buffer. The pH disclosed in this reference is 7.4.

WO2005/097094 discloses a composition for topical administration (i.e. administration to the skin of a human or animal) of an antimicrobial agent in a formulation with a chelating agent and a buffer. The pH of the solution is disclosed between 3 and 9, but preferably between 5 and 8. Specific formulations have a pH no lower than 6.7 and are primarily alkaline.

Shelf life is an important issue for pharmaceuticals that treat ocular infection. Particularly, no less than 90% of an active agent can deteriorate over a two-year period of time to be approved by the Food and Drug Administration. Biguanides are somewhat unstable and degrade in an aqueous solution.

Consequently, there is a need for a stable ophthalmic antimicrobial composition that is relatively non-toxic and effective against a wide range of microbes. The present invention addresses this and other needs.

### SUMMARY OF THE INVENTION

The present invention includes, in one aspect, a composition for treating infectious disease. The composition comprises an ophthalmically acceptable aqueous composition. The ophthalmically acceptable aqueous composition comprises water, a biguanide containing antimicrobial agent and a pH adjusting agent. The pH adjusting agent adjusts the composition to a storage pH that is a minimum of 4 and a maximum of 6. Upon, instillation of the composition in the eye, the pH increases to physiological pH resulting in a composition at physiological pH with enhanced antimicrobial efficacy relative to the composition at storage pH.

The present invention includes, in one embodiment, a method of treating a patient having infectious disease. The method comprises administering an ophthalmically acceptable aqueous solution to the ocular region of a patient. The method includes treating, fungal infection, amoebal infection, including amoebal keratitis, viral infection and bacterial infection (including bacterial conjunctivitis).

In one embodiment, the solution optionally comprises a buffer.

It is preferable, in one embodiment, to reach physiological pH rapidly after instillation in the eye. Typically, the physiological pH is obtained after 5 seconds, 4 seconds, 3 seconds, 2 seconds or 1 second. Blinking often assists in mixing the drops with the tear film and removing excess fluid. Typically, the physiological pH is obtained after 5 blinks. Typically, the physiological pH is obtained after 4 blinks, 3 blinks, 2 blinks or 1 blink.

In one embodiment, the composition at storage pH has an enhanced shelf-life relative to the shelf-life of the composition at physiological pH. Generally, the composition at storage pH has a shelf life that is a minimum of about 5%, about 10%, about 15%, about 20% longer than the shelf life at physiological pH.

### DESCRIPTION OF DRAWINGS

FIG. 1 is the percentage of Alexidine remaining in various pH adjusted solutions stored in glass vials at 25 C for two weeks.

FIG. 2 shows the concentration versus time profiles for Alexidine in the rabbit.

### DETAILED DESCRIPTION

A composition for treating infectious disease comprising an ophthalmically acceptable aqueous composition, the ophthalmically acceptable aqueous composition comprises water, a biguanide containing antimicrobial agent and a pH adjusting agent to adjust the composition to a storage pH that is a minimum of 4 and a maximum of 6, wherein upon instillation of the eye, the pH increases to physiological pH resulting in a composition at physiological pH with enhanced antimicrobial efficacy relative to the composition at storage pH.

When the composition is instilled in the eye a pH shift is observed to physiological pH. Preferably, the physiological pH is obtained rapidly, wherein the physiological pH is obtained after 5 seconds. Typically, the physiological pH is obtained after 4 seconds, 3 seconds, 2 seconds, or 1 second.

The physiological pH is obtained after 5 blinks. Typically, the physiological pH is obtained after 4 blinks, 3 blinks, 2 blinks or 1 blink.

The method of claim 1, wherein the solution at storage pH has an enhanced shelf-life relative to the shelf-life of the solution at physiological pH.

The method of claim 1, wherein the solution at storage pH has a shelf life that is 5% longer than the shelf life at physiological pH.

Alexidine is a biguanide antimicrobial agent that is defined by the formula 1,1′-hexamethylene-bis[5-(2-ethylhexyl)biguanide]. By biguanide antimicrobial agent it is meant an antimicrobial agent that has biguanide substituents and has antimicrobial properties in an ophthalmically safe amount. Suitable biguanide antimicrobial agents include but are not limited to 1,1′-hexamethylene-bis[5-(p-chlorophenyl)biguanide] (Chlorhexidine) or water soluble salts thereof, 1,1′-hexamethylene-bis[5-(2-ethylhexyl)biguanide] (Alexidine) or water-soluble salts thereof, and poly(hexamethylene biguanide) (PHMB).

In one embodiment, the amount of antimicrobial agent in the ophthalmic composition is a maximum of about 1 ppm and a minimum of about 0.1 wt. %. Typically, the amount of antimicrobial agent in the multipurpose solution is a minimum of about 4.5 ppm, about 5 ppm, about 10 ppm, about 15 ppm or about 20 ppm. Typically, the amount of antimicrobial agent in the ophthlamic solution is a maximum of about 1000 ppm, about 500 ppm, about 300 ppm, about 100 ppm, about 75 ppm or about 50 ppm.

In one embodiment, the amount of antimicrobial agent is sufficient to provide an in eye concentration that is a minimum of about 0.001 ppm and a maximum of about 100 ppm. Preferably, the in-eye concentration is a minimum of about 0.1 ppm, about 1 ppm, about 5 ppm, about 10 ppm or about 50 ppm and is a maximum of about 90 ppm, about 80 ppm, about 70 ppm or about 60 ppm. The in-eye concentration is determined by sampling a portion of the lacrimal fluid after 3 blinks of the eye. The in-eye concentration is based upon the concentration of antimicrobial agent. It may also be significantly affected by the concentration of viscosity adjusting agents, penetration enhancers, surfactants, if any, and other agents that may cause the antimicrobial agent to remain in solution or bind to the corneal tissue of the eye. In one embodiment, the in-eye concentration of antimicrobial agent is 10% of the concentration of antimicrobial agent in the storage solution.

The penetration enhancer generally acts to make the cell membranes less rigid and therefore more amenable to allowing passage of drug molecules between cells. The penetration enhancers preferably exert their penetration enhancing effect immediately upon application to the eye and maintain this effect for a period of approximately five to ten minutes. The penetration enhancers and any metabolites thereof must also be non-toxic to ophthalmic tissues. One or more penetration enhancers will generally be utilized in a minimum amount of about 0.01 weight percent and/or a maximum of about 10 wt. %.

The preferred penetration enhancers are saccharide surfactants, such as dodecylmaltoside (“DDM”), and monoacyl phosphoglycerides, such as lysophosphatidylcholine. The saccharide surfactants and monoacyl phosphoglycerides, which may be utilized, as penetration enhancers in the present invention are known compounds. The use of such compounds to enhance the penetration of ophthalmic drugs is described in U.S. Pat. No. 5,221,696 the entire contents of which are incorporated by reference into the present specification. Other penetration enhancers that are ophthalmically acceptable include EDTA and other Ca2+ chelating agents, cytochalasin B (a group of small molecules bind specifically to actin microfilaments), cyclodextrin and derivatives.

Due to the tendency of Alexidine or other biguanide antimicrobial agents to hydrolyze in an aqueous solution, it is desirable to optionally include a stabilizer. The present invention enhances the stability of the composition for long-term storage and in one embodiment requires no stabilizer. However, a stabilizer can be added to further boost the stability. The present invention, when used in the presence of a stabilizer will further extend the shelf-life of the composition.
A stabilizer is a compound that prevents the chemical degradation of an active agent in solution. Examples of stabilizers that are effective in an aqueous solution include but are not limited to ophthalmically acceptable antioxidants (ascorbate, methionine, citric acid, BHT), complexing agents (cyclodextrin and derivatives, hyaluronic acid, citric acid), non-ionic surfactants (poloxamines such as Tetronics® 908, tyloxapol) and chelating agents and salts thereof (hydroxyl alkyl phosphonate, sodium edentate).

In one embodiment, the stabilizer is present in an amount effective to stabilize the biguanide compound at a pH that is a minimum of 4 and a maximum of 6. An amount effective to stabilize a compound means that the stabilizer is present in an amount that prevents deterioration of at least 90% of the compound in a period of 24 months when stored at a pH that is a minimum of 4 and a maximum of 6. In another embodiment, the preferred stabilizer is present in a minimum amount of about 0.001 wt. %, about 0.005 wt. %, about 0.01 wt. % and/or a maximum amount of about 0.5 wt. %, about 0.3 wt. %, about 0.1 wt. %, about 0.08 wt. %, about 0.05 wt. %, about 0.03 wt. %, about 0.01 wt. %.

In another embodiment, the effective shelf life of the antimicrobial agent is extended by a minimum of about 10 percent of the shelf life without the stabilizer. In another embodiment, the antimicrobial agent is extended by a minimum of about 20 percent, about 40 percent, about 80 percent, about 100 percent or about 200 percent.

Delivery Vehicle

In another embodiment, the solution of the present invention contains a delivery vehicle that increases the mean residence time of the active agent in the eye and/or enhances penetration in the eye. U.S. Pat. No. 6,884,788 or 6,261,547 or 5,800,807 or 5,618,800 or 5,496,811 disclose various ophthalmic delivery vehicles the teachings in these patents are incorporated by reference in their entirety.

Various anatomical barriers relating to the eye may underlie the poor intraocular penetrance of active ingredients. In this regard, the cornea is the principal barrier to entry of foreign substances. It has two distinct penetration barriers, the corneal epithelium and the corneal stroma. Thus, it is desirable to use a penetration enhancer to improve the penetration of the active ingredients of the present invention.

The penetration enhancer generally acts to make the cell membranes less rigid and therefore more amenable to allowing passage of drug molecules between cells. The penetration enhancers preferably exert their penetration enhancing effect immediately upon application to the eye and maintain this effect for a period of approximately five to ten minutes. The penetration enhancers and any metabolites thereof must also be non-toxic to ophthalmic tissues. One or more penetration enhancers will generally be utilized in a minimum amount of about 0.01 weight percent and/or a maximum of about 10 wt. %.

The preferred penetration enhancers are saccharide surfactants, such as dodecylmaltoside (“DDM”), and monoacyl phosphoglycerides, such as lysophosphatidylcholine. The saccharide surfactants and monoacyl phosphoglycerides, which may be utilized, as penetration enhancers in the present invention are known compounds. The use of such compounds to enhance the penetration of ophthalmic drugs is described in U.S. Pat. No. 5,221,696 the entire contents of which are incorporated by reference into the present specification.

The viscosifiers are optionally used in the present invention to increase the mean residence time of the active ingredient in the eye. With the aid of a viscosifier, liquid drops can be used having a viscosity that is a minimum of about 2 cps and a maximum of about 100 cps. Viscosifiers can be used to formulate liquid gels that have a viscosity ranging from about 100 cps to about 1000 cps. Ophthalmic gels will generally have a viscosity in excess of 1,000 cps. Regardless, the viscosifier is utilized to ensure an adequate mean residence time in the eye. Any synthetic or natural polymer, which is capable of forming a viscous or a solid insert, may be utilized. In addition to having the physical properties required to form a viscous gel or solid insert, the polymers must also be compatible with tissues of the eye. The polymers must also be chemically and physically compatible with the above-described active agent and other components of the compositions.

Polymers, which satisfy the foregoing criteria, are referred to herein as “ophthalmically acceptable viscous polymers.” Examples of suitable polymers include: natural polysaccharides and gums, such as alginate, carrageenan, guar, karaya, locust bean, tragacanth agarose and xanthan; modified naturally occurring polymers such as carboxymethylcellulose, hydroxyethylcellulose, hydroxypropylcellulose, hydroxypropylmethylcellulose, methylcellulose, hydroxypropylmethylguar and carboxymethyguar, synthetic polymers, such as carboxy vinyl polymers, polyvinyl alcohol and polyvinyl pyrrolidone.

In addition, proteins and synthetic polypeptides that form viscous gels and are ophthalmically acceptable can be used to provide better bioavailability. Typically, proteins that can be used include: gelatin, collagen, albumin, whey protein and casein.

Polymers which have high molecular weights and, most importantly, physical properties that mimic the physical properties of the mucous secretions found in the eye are referred to herein as being “mucomimetic.” A preferred class of mucomimetic polymers are carboxy vinyl polymers having molecular weights in the range of from about 50,000 to about 6,000,000. The polymers have carboxylic acid functional groups and preferably contain between 2 and 7 carbon atoms per functional group. The gels that form during preparation of the ophthalmic polymer dispersion have a viscosity between about 1,000 to about 300,000 centipoise (cps). Suitable carboxy vinyl polymers include those called carbomers, e.g., Carbopol[R] (B. F. Goodrich Co., Cleveland, Ohio). Specifically preferred are carbomer 934, 940, 970, 974 and 980. Such polymers will typically be employed in an amount between about 0.05 and about 8.0 wt %, depending on the desired viscosity of the composition.

The composition comprises a pH adjusting agent. A pH adjusting agent is an acid, base or salt that is added to the solution primarily to adjust the pH of the solution. However, it is understood that some known acids, buffers and salts have been found to have antimicrobial properties, enhance antimicrobial properties and serve other functions. They are predominantly used in the art to adjust pH of a solution. Some acids that may be used to adjust the pH include hydrocloric acid and the acid buffers listed below. Some bases that are used to adjust pH include sodium hydroxide, potassium hydroxide, and the basic buffers listed below.

In one embodiment, the pH adjusting agent is a buffer or buffer combination. One or more conventional buffers can alternatively be employed to obtain the desired pH value. Suitable buffers include, but not limited to, acetate buffer, citrate buffer, formate buffer, histidine, succinate buffer, phosphate buffer, maleate buffer, propionate buffer, malate buffer, pyridine buffer, piperazine buffer, cacodylate buffer, MES buffer, bis-tris buffer, carbonate buffer, imidazole buffer, ADA buffer, ACES buffer, PIPES buffer, MOPSO buffer, HEPES buffer, MOPS buffer, BES buffer, triethanolamine buffer, triethanolamine buffer and borate buffer. Generally, buffers will be used in amounts ranging from about 0.05 to about 2.5 weight percent, and preferably, from about 0.1 to about 1.5 weight percent. Preferably, the buffer will have a pKa that is from about 3.0 to about 8.0. Preferably, the buffer will have a pKa that is a minimum of 4.5 or 5.0 or 5.5 and/or a maximum of about 7.0, about 6.5 or about 6.0.

In the storage solution or storage composition, the pH will be a minimum of about 4 and a maximum of about 6. Preferably, the pH will be a minimum of about 4.5, about 4.7, about 5, about 5.1, about 5.2, about 5.3 and a maximum of about 6.0, about 5.9, about 5.8, about 5.7, about 5.6 or about 5.5. Preferably, the pH is 5.5. It is advantageous that the buffer be present in an amount that will maintain a stable pH during the shelf-life of the product. However, if the selection of the buffer and its amount is such that the buffering capacity is too high, the pH may not rapidly affect a shift in pH upon instillation in the eye without excessive dilution of the solution.

Compositions of the present invention likewise include one or more tonicity agents to approximate the osmotic pressure of normal lachrymal fluids, which is equivalent to a 0.9 percent solution of sodium chloride or 2.5 percent glycerin solution. Examples of suitable tonicity agents include but are not limited to sodium and potassium chloride, dextrose, mannose, glycerin, calcium and magnesium chloride. These agents are typically used individually in amounts that are a minimum of about 0.01 wt. % or about 0.2 wt. % and/or a maximum of about 2.5 wt. % or 1.5 wt. %. Preferably, the tonicity agent is employed in an amount to provide a final osmotic value that is a minimum of 200 mOsm/kg, 220 mOsm/kg and/or a maximum of about 450 mOsm/kg, 350 mOsm/kg or about 320 mOsm/kg.

Aqueous compositions may likewise include a humectant to provide moisture to the eye. A first class of humectants is polymer humectants. Examples of suitable humectants include for example but are not limited to poly(vinyl alcohol) (PVA), poly(N-vinylpyrrolidone) (PVP), cellulose derivatives and poly(ethylene glycol). As disclosed in U.S. Pat. No. 6,274,133, cationic cellulosic polymers include for example but are not limited to water soluble polymers commercially available under the CTFA (Cosmetic, Toiletry, and Fragrance Association) designation Polyquaternium-10, including the cationic cellulosic polymers available under the trade name UCARE® Polymers from Amerchol Corp., Edison, N.J., such as for example but not limited to Polymer JR™. Generally, these cationic cellulose polymers contain quaternized N,N-dimethylamino groups along the cellulosic polymer chain.

Another suitable class of humectants is non-polymeric humectants. Examples may include glycerin, propylene glycol, and other non-polymeric diols and glycols. The specific quantities of humectants used in the invention will vary depending upon the application. However, the humectants will typically be included in an amount from about 0.01 to about 5 weight percent, preferably from about 0.1 to about 2 weight percent.

It will be understood that some constituents possess more than one functional attribute. For example, cellulose derivatives are suitable polymeric humectants, but are also referred to as “viscosity increasing agents” to increase viscosity of the composition if desired. Glycerin is a suitable non-polymeric humectant but is also may contribute to adjusting tonicity.

Compositions of the present invention may optionally include one or more sequestering agents to bind metal ions, which in the case of ophthalmic solutions, might otherwise react with protein deposits and collect on contact lenses. Suitable sequestering agents include for example but are not limited to ethylenediaminetetraacetic acid (EDTA) and its salts. Sequestering agents are preferably present in a minimum of about 0.01 wt. % and/or a maximum of about 0.2 wt. %.

It will be understood that the present invention is typically applied by administering a solution to the eye of a patient in the form of eye drops, liquid gels or viscous gels. In one embodiment, one to four drops are applied to each eye. Preferably two drops are applied to each eye. In one embodiment, the drops are placed directly on the eye. In another embodiment, the drops are placed in the conjunctival sac beneath the eye.

Typically, the drops are administered a minimum of once daily, two times daily, three times daily, or four times daily and a maximum of once hourly, once every two hours, once every three hours, once every four hours, once every six hours.

### EXAMPLES

### Example 1

### Formulation 1

The following ingredients were combined to produce a solution identified as Formulation 1. Formulation 1, upon instillation in the eye rapidly adjusted to physiological pH.

TABLE 1
Formulation 1

| Ingredient | w/w(%) |
|---|---|
| Alexidine | 100 ppm |
| HPMC | 0.5 |
| Pluronic F127 | 0.2 |
| Mannitol | 5.0 |
| EDTA | 0.1 |
| Citrate Buffer | pH 5.0 |
| WFI | |

### Example 2

### Formulation 2

The following ingredients were combined to produce a solution identified as Formulation 2. Formulation 2, upon instillation in the eye rapidly adjusted to physiological pH.

TABLE 2
Formulation 2

| Ingredient | w/w(%) |
|---|---|
| Alexidine | 100 ppm |
| PVP K90 | 2.0 |
| Pluronic F127 | 0.15 |
| Tyloxapol | 0.1 |
| EDTA | 0.05 |
| Acetate Buffer | pH 5.0 |
| WFI | |

### Example 3

### Formulation 3

The following ingredients were combined to produce a solution identified as Formulation 3. Formulation 3, upon instillation in the eye rapidly adjusted to physiological pH.

TABLE 3
Formulation 3

| Ingredient | W/W(%) |
|---|---|
| Alexidine | 200 ppm |
| HPMC | 1.0 |
| EDTA | 0.15 |
| Methionine | 0.25 |
| Tyloxapol | 0.05 |
| Mannitol | 3.0 |
| Histidine | pH 5.5 |
| WFI | |

### Example 4

### The pH Profile of Alexidine at 20 ppm after Two-Week Incubation at 45° C., 20% Humidity

The purpose of this study was to identify optimal pH for Alexidine. Five solutions of 20 ppm Alexidine were formulated in borate buffer solutions. Each formulation was pH adjusted using NaOH or HCl, as follows:

TABLE 4
pH of Alexidine Formulations in Example 4

| Group | pH of Sample |
|---|---|
| Group 1 | 3.0 |
| Group 2 | 5.0 |
| Group 3 | 7.0 |
| Group 4 | 9.0 |
| Group 5 | 11.0 |

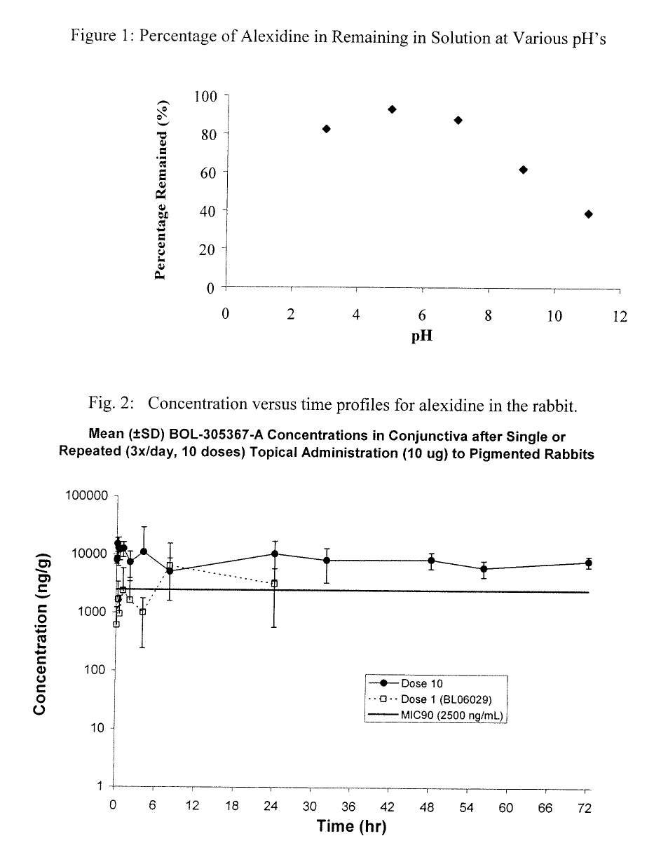

The samples were placed in glass containers and maintained at 40° C. and a humidity of 20% for a period of two weeks. At the end of two weeks, the samples were tested to determine how much Alexidine was present in solution. The results are illustrated in FIG. 1. It was concluded that the most stable range of pH for Alexidine was between about pH 4 and pH 6.

### Example 5

### The pH Effect on the Loss of Alexidine Due to Non-Specific Binding

Alexidine sample solutions (20 ppm) were prepared at different pHs and quantitative amount were stored in glass vial for two weeks. At the end of storage, the sample solution was removed and thoroughly rinsed with distilled water. The empty sample vials were then rinsed with 0.1 N HCl solution. The concentration of Alexidine in the acid solution was determined by HPLC assay and the amount recovered from glass wall was calculated in Table 1. It suggests that non-specific binding of Alexidine to the glass wall increases when pH increases.

TABLE 5
Amount of Alexidine recovered from
glass vial at different pH values

pH

| | 3 | 5 | 7 | 9 | 11 |
|---|---|---|---|---|---|
| % Adsorbed on Glass Vial | 1.0 | 4.0 | 7.5 | 12.5 | 23.5 |

### Example 6

### Animal PK Studies for Alexidine Solution Formulations

This study was to investigate anterior ocular and systemic pharmacokinetics of alexidine following repeated topical ocular administration (3 doses/day; 10 doses total) of a 100 ppm solution to male New Zealand Composite rabbits.

This study was carried out using a non-crossover design and a total of 52 naïve male New Zealand Composite rabbits. Each animal (fed) received a topical ocular dose of alexidine at approximately 7 am, 12 noon and 5 pm on Day 1, Day 2 and Day 3. On the morning of Day 4, each animal received the final (10^{th}) topical ocular dose. For each dose administration, animals received approximately 100 μL of a 100 ppm solution (10 μg dose) as a topical instillation into the conjunctival sac of each eye using a positive-displacement pipette.

On Day 4, rabbits (n=4 per collection time) were euthanized and the eyes enucleated and dissected. Samples of blood (centrifuged to obtain plasma), tear, conjunctiva, cornea, and aqueous humor were obtained from both eyes of each animal. Samples were collected prior to administration of the final dose, and 5, 15, and 30 min and 1, 2, 4, 8, 24, 32, 48, 56, and 72 hr after dosing. Non-compartmental methods were used for pharmacokinetic analysis of concentration versus time data. Due to the destructive sampling regimen employed in this study average composite data were used in the pharmacokinetic analysis. The pharmacokinetic parameters are set forth in Table 6:

TABLE 6
Pharmacokinetic Paramaters of Alexidine

| Dose | | Cₘₐₓ | Tmax | AUC₍₀₋₂₄₎ | DNAUC₍₀₋₂₄₎ |
|---|---|---|---|---|---|
| Regimen | Matrix | (ng/g) | (min) | (min · ug/g) | (min · ug/g) ÷ ug |
| TID Dosing | Conjunctiva | 15144 ± 4118 | 5 | 11461 | 1146 |
| (Dose 10) | Cornea | 3737 ± 1794 | 30 | 2727 | 273 |
| 10 μg/eye | Aq. Humor | Analysis Ongoing | | | |
| | Plasma | Analysis Ongoing | | | |
| Single Dose | Conjunctiva | 6440 | 480 | 5697 | 570 |
| 10 μg/eye | Cornea | 997 | 15 | 557 | 55.7 |

| | | |
|---|---|---|
| (BL06029) | Aq. Humor | No Quantifiable levels (<0.5 ng/mL) |
| | Plasma | No Quantifiable levels (<0.5 ng/mL) |

Abbreviations used in this diagram are as follows. Cmax is defined as the maximum mean concentration attained after topical instillation (±SD). Tmax is the time that Cmax was observed. AUC₍₀₋₂₄₎ is defined as the area under the matrix concentration versus time curve from the time of dosing through 24 hour post-dose. DNAUC is the dose-normalized area under the matrix concentration versus time curve from the time of dosing through 24 hour post-dose (calculated as AUC₍₀₋₂₄₎/dose (μg/eye)).

The mean concentrations of alexidine in conjunctiva was about 5100 ng/g, or higher, at all times through the 72 hr after the final 10-μg dose. This data suggest that TID dosing with a 10 μg dose is sufficient to maintain consistent coverage above MIC90 for alexidine. The Cmax:MIC90 ratio for alexidine, with repeated administration of a 10-μg dose, was about 6 to about 8. The AUC:MIC90 ratio for alexidine, with repeated administration of a 10-μg dose, this ratio was about 76 to about 96 (using an AUC₍₀₋₂₄₎ value of 191,016 ng*hr/mL). In the present study, repeated administration of a 100 ppm solution was well tolerated in all animals.

What is claimed is:

1. A method of treating infectious disease comprising administering an ophthalmically acceptable aqueous solution to the ocular region of a patient, the ophthalmically acceptable aqueous solution comprising water, a biguanide containing antimicrobial agent comprising and a pH adjusting agent to adjust the solution to a storage pH to a minimum of 4 and a maximum of 6, wherein upon instillation of the eye, the pH increases to physiological pH resulting in a solution at physiological pH with enhanced antimicrobial efficacy relative to solution at storage pH.

2. The method of claim 1, wherein the biguanide antimicrobial agent is selected from the group consisting of poly(hexamethylene biguanide), alexidine and chlorhexidine, and salts thereof.

3. The method of claim 1, wherein the biguanide antimicrobial agent is alexidine, and salts thereof.

4. The method of claim 1, wherein the amount of alexidine is a minimum of about 0.1 ppm and a maximum of about 5000 ppm.

5. The method of claim 1, wherein the amount of alexidine is sufficient to provide an in eye concentration that is a minimum of about 0.001 ppm and a maximum of about 100 ppm.

6. The method of claim 1, wherein the solution further comprises a penetration enhancer.

7. The method of claim 1, wherein the penetration enhancer is present in an amount that is a minimum of about 0.001 wt. % and a maximum of about 5 wt. %.

8. The method of claim 1, wherein the solution at storage pH has an enhanced shelf-life relative to the shelf-life of the solution at physiological pH.

9. The method of claim 1, wherein the solution at storage pH has a shelf life that is at least 5% longer than the shelf life at physiological pH.

10. The method of claim 1, wherein the solution further comprises a viscosifier.

11. The method of claim 1, wherein the viscosifiers are selected from the group consisting of natural polysaccharides, natural gums, modified natural polymers, synthetic polymers, proteins and synthetic polypeptides that are capable of increasing viscosity and are ophthalmically acceptable.

12. The method of claim 1, wherein the viscosifiers are selected from the group consisting of mucomimetics.

13. The method of claim 1, wherein the viscosifier is a carboxyvinyl polymer.

14. The method of claim 1, wherein the biguanide antimicrobial agent is present in an amount ranging from 0.1 ppm to about 5.0 wt. % based upon the total amount the solution.

15. The method of claim 1, wherein the infectious disease is a fungal infection.

16. The method of claim 1, wherein the infectious disease is an amoeba infection.

17. The method of claim 16, wherein the amoeba infection is amoebal keratitis.

18. The method of claim 1, wherein the infectious disease is a viral infection.

19. The method of claim 1, wherein the infectious disease is a bacterial infection.

20. The method of claim 19, wherein the infectious disease is bacterial conjunctivitis.

21. The method of claim 1, wherein the solution further optionally comprises a buffer having a pKa that is from about 3.0 to about 8.0.

22. The method of claim 1, wherein the buffer is selected from the group consisting of acetate buffer, citrate buffer, formate buffer, histidine, succinate buffer, phosphate buffer, maleate buffer, propionate buffer, malate buffer, pyridine buffer, piperazine buffer, cacodylate buffer, MES buffer, bis-tris buffer, carbonate buffer, imidazole buffer, ADA buffer, ACES buffer, PIPES buffer, MOPSO buffer, HEPES buffer, MOPS buffer, BES buffer, triethanolamine buffer, triethanolamine buffer and borate buffer.

23. The method of claim 1, wherein the physiological pH is obtained after 5 seconds. Typically, the physiological pH is obtained after 4 seconds, 3 seconds, 2 seconds, or 1 second.

24. The method of claim 1, wherein the physiological pH is obtained after 5 blinks. Typically, the physiological pH is obtained after 4 blinks, 3 blinks, 2 blinks or 1 blink.

24. A composition for treating infectious disease comprising an ophthalmically acceptable aqueous composition, the ophthalmically acceptable aqueous composition comprises water, a biguanide containing antimicrobial agent and a pH adjusting agent to adjust the composition to a storage pH that is a minimum of 4 and a maximum of 6, wherein upon instillation of the eye, the pH increases to physiological pH resulting in a composition at physiological pH with enhanced antimicrobial efficacy relative to the composition at storage pH.

25. The composition of claim 24, wherein the biguanide antimicrobial agent is selected from the group consisting of poly(hexamethylene biguanide), alexidine and chlorhexidine.

26. The composition of claim 1, wherein the biguanide antimicrobial agent is alexidine.

27. The composition of claim 1, wherein the amount of alexidine is a minimum of about 0.1 ppm and a maximum of about 5000 ppm.

28. The composition of claim 24, wherein the amount of alexidine is sufficient to provide an in eye concentration that is a minimum of about 0.001 ppm and a maximum of about 100 ppm.

29. The composition of claim 24, wherein the composition further comprises a penetration enhancer.

30. The composition of claim 24, wherein the penetration enhancer is present in an amount that is a minimum of about 0.001 wt. % and a maximum of about 5 wt. %.

31. The composition of claim 24, wherein the composition at storage pH has an enhanced shelf-life relative to the shelf-life of the composition at physiological pH.

32. The composition of claim 24, wherein the composition at storage pH has a shelf life that is at least 5% longer than the shelf life at physiological pH.

33. The composition of claim 24, wherein the composition further comprises a viscosifier.

34. The composition of claim 24, wherein the viscosifiers are selected from the group consisting of natural polysaccharides, natural gums, modified natural polymers, synthetic polymers, proteins and synthetic polypeptides that are capable of increasing viscosity and are ophthalmically acceptable.

35. The composition of claim 24, wherein the viscosifiers are selected from the group consisting of mucomimetics.

36. The composition of claim 24, wherein the viscosifier is a carboxyvinyl polymer.

37. The composition of claim 24, wherein the biguanide antimicrobial agent is present in an amount ranging from 0.1 ppm to about 5.0 wt. % based upon the total amount the composition.

38. The composition of claim 24, wherein the infectious disease is a fungal infection.

39. The composition of claim 24, wherein the infectious disease is an amoeba infection.

40. The composition of claim 39, wherein the amoeba infection is amoebal keratitis.

41. The composition of claim 24, wherein the infectious disease is a viral infection.

42. The composition of claim 24, wherein the infectious disease is a bacterial infection.

43. The composition of claim 42, wherein the infectious disease is bacterial conjunctivitis.

44. The composition of claim 24, wherein the composition further optionally comprises a buffer having a pKa that is from about 3.0 to about 8.0.

45. The composition of claim 24, wherein the buffer is selected from the group consisting of acetate buffer, citrate buffer, formate buffer, histidine, succinate buffer, phosphate buffer, maleate buffer, propionate buffer, malate buffer, pyridine buffer, piperazine buffer, cacodylate buffer, MES buffer, bis-tris buffer, carbonate buffer, imidazole buffer, ADA buffer, ACES buffer, PIPES buffer, MOPSO buffer, HEPES buffer, MOPS buffer, BES buffer, triethanolamine buffer, triethanolamine buffer and borate buffer.

46. The composition of claim 24, wherein the physiological pH is obtained after 5 seconds. Typically, the physiological pH is obtained after 4 seconds, 3 seconds, 2 seconds, or 1 second.

47. The composition of claim 24, wherein the physiological pH is obtained after 5 blinks. Typically, the physiological pH is obtained after 4 blinks, 3 blinks, 2 blinks or 1 blink.
